(19) United States
(12) Patent Application Publication
(10) Pub. No.: US 2018/0130549 A1
(43) Pub. Date: May 10, 2018

(54) FAMILY GENETIC HISTORY STORAGE SYSTEM FOR STORING DNA INFORMATION ON AN IDENTIFICATION CERTIFICATE

(71) Applicant: Larry FUSON, LaPorte City, IA (US)

(72) Inventor: Larry FUSON, LaPorte City, IA (US)

(21) Appl. No.: 15/864,295

(22) Filed: Jan. 8, 2018

Related U.S. Application Data
(63) Continuation-in-part of application No. 14/160,406, filed on Jan. 21, 2014, now abandoned.
Application No. 14/160,406 is a continuation-in-part of application No. 13/556,138, filed on Jul. 23, 2012, now abandoned.
Application No. 13/556,138 is a continuation-in-part of application No. 13/166,965, filed on Jun. 23, 2011, now abandoned.
(60) Provisional application No. 61/357,537, filed on Jun. 23, 2010.

(51) Int. Cl. G16H 10/60; G16H 10/40; A61B 5/15; A61B 5/00

(52) U.S. Cl. CPC G16H 10/60; A61B 5/448; A61B 5/1411; G16H 10/40

(57) ABSTRACT

A family genetic history storage system for storing DNA information on an identification certificate includes a selected area for attaching an identification indicators with at least one DNA blood sample storage area. It includes a means for storing the blood sample for future use and analysis while protecting it from environmental contamination. The user may be provided with DNA blood sample filter paper and a disposable lancet to help the user to easily obtain a blood sample. In another embodiment at least one DNA storage container is provided to allow for storage of biological cell material samples such as hair or fingernails. The certificate also may include an official certification proof area where official provenance is attached to certify authenticity.

### CROSS REFERENCE TO RELATED APPLICATIONS

This application is a continuation in part of U.S. patent application Ser. No. 14/160,406 filed Jan. 21, 2014, which is a continuation in part of U.S. patent application Ser. No. 13/556,138, filed Jul. 23, 2012, which is a continuation in part of U.S. patent application Ser. No. 13/166,965, filed Jun. 23, 2011, which in turn claims priority to Provisional Application No. 61/357,537, filed on Jun. 23, 2010, the complete disclosures of each of which are hereby incorporated by reference.

### BACKGROUND OF THE INVENTION

Keeping track of a person's identity is becoming more important in today's society. However, even in historical times, being able to prove parentage was sometimes an issue as evidenced by the famous story of king Solomon judging between two woman both claiming to be the mother. The wise king was able to discern the truth, but short of being the wisest human, science has provided us with many tools to determine identity.

As we continue to make progress regarding genetic medicine with the associated treatments and testing that is available, it is even more important to be able to trace genetic information reaching back many generations. Often this information is difficult to determine as family members die and are no longer available to interview and test. There is a need for a method of storing and displaying family genetic histories in a safe and reliable manner to enhance the medical treatments that are now available and that will be available in the future.

Even with many safeguards and protocols in place, it is not unknown to have a hospital mix up babies and parents are sent home with the wrong children. Science has provided us with tools such as blood types to try to determine those that could not be the parents: it has only recently become possible to show with great certainty parentage. DNA testing is becoming more common and is often employed forensically in criminal cases.

There is a need for a positive identification instrument that relies on the science of DNA to prove identity that is inexpensive, easy to use and promotes long term viability of the DNA sample and allows a user to store the information digitally.

### SUMMARY OF THE INVENTION

A family genetic history storage system for storing DNA information on an identification certificate includes a selected area for attaching an identification indicators with at least one DNA blood sample storage area. It includes a means for storing the blood sample for future use and analysis while protecting it from environmental contamination. The user may be provided with DNA blood sample filter paper and a disposable lancet to help the user to easily obtain a blood sample. In another embodiment at least one DNA storage container is provided to allow for storage of biological cell material samples such as hair or fingernails. The certificate also may include an official certification proof area where official provenance is attached to certify authenticity.

Other features and advantages of the instant invention will become apparent from the following description of the invention which refers to the accompanying drawings.

### BRIEF DESCRIPTION OF THE DRAWINGS

FIG. 1 is an illustration of a DNA enabled birth certificate according to an embodiment of the invention.

FIG. 2 is an illustration of the DNA enabled birth certificate shown in FIG. 1 with an alternative DNA enabled storage system and biometric.

FIG. 3 is an illustration of the DNA enabled birth certificate shown in FIG. 1 with another alternative DNA storage system.

FIG. 4 is an illustration of a DNA enabled identity certificate according to an embodiment of the invention.

FIG. 5 is an illustration of a DNA enabled identification card according to an embodiment of the invention.

FIG. 6 is an illustration of a DNA enabled identification card attached to a chain according to an embodiment of the invention.

FIG. 7 is an illustration of a biometric birth certificate with a DNA sequencing chip mounted therein.

FIG. 8A is an illustration of the biometric birth certificate with a USB interface and computer storage in a enabled position.

FIG. 8B is an illustration of the biometric birth certificate with a USB interface and computer storage in a storage position.

FIG. 9 is an illustration of the biometric birth certificate with a computer DNA printout section.

FIG. 10 is an illustration of a biometric birth certificate with a RFID chip.

FIG. 11 is an illustration of a biometric birth certificate according to an embodiment of the invention.

FIG. 12 is a flow diagram of a family genetic history storage system and method for storing and displaying DNA information according to an embodiment of the present invention.

FIG. 13 is an illustration of a birth certificate and family genetic history storage system sample holder according to an embodiment of the present invention.

FIG. 14 is a system diagram of a birth certificate and family genetic history storage system sample holder using a wireless connection according to an electronic embodiment of the present invention.

FIG. 15 is a system diagram of a birth certificate and family genetic history storage system sample holder using a wired connection according to an electronic embodiment of the present invention.

FIG. 16 is a system diagram of a birth certificate and family genetic history storage system sample holder using a cloud based wireless connection according to an electronic embodiment of the present invention.

FIG. 17 is an illustration of the DNA enabled birth certificate having a sealable DNA sample portion and an official proof section.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

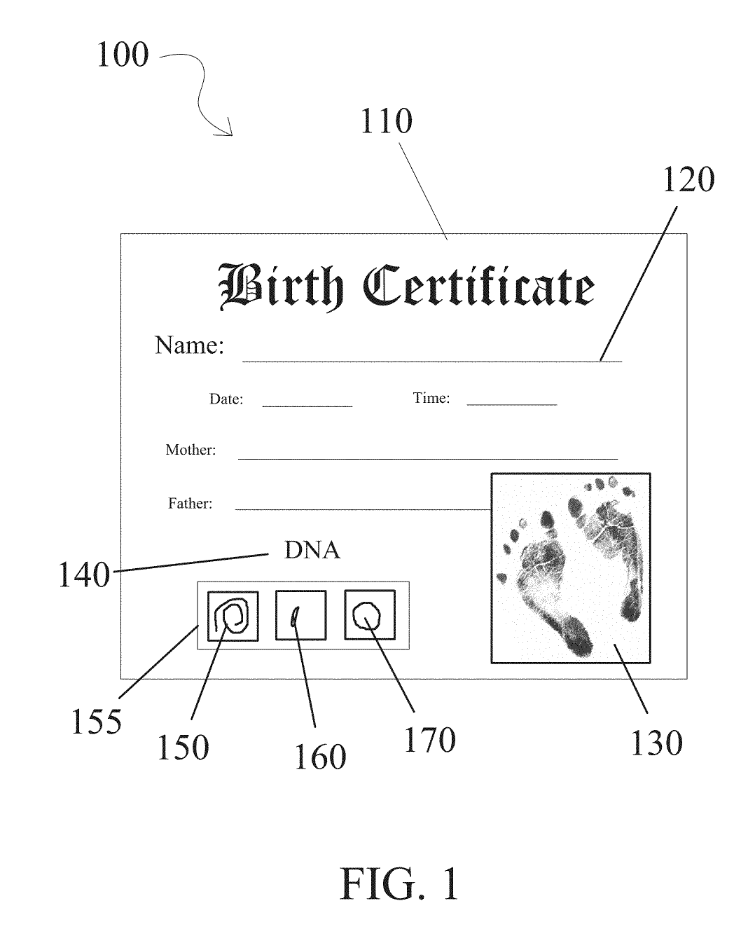

Referring to FIG. 1, a DNA birth certificate 100 is shown having a certificate 110 with printed information 120 such as name, date and time of birth along with parental information. Of course other information may be included or excluded since the information shown is merely representative of common kinds of information found on birth certificates. In addition to written information 120, graphical information such as footprints 130 may be applied.

A DNA sample area 140 is provided to allow a user to secure DNA containing samples to DNA birth certificate 100. Many kinds of biologically active samples may be used such as hair 150, nail clipping 160 and blood 170. These samples are sealed within a laminated section 155 to preserve the samples. Of course any biologically active sample may be used as long as it is practical to fit within the area provided 155 and contains intact DNA.

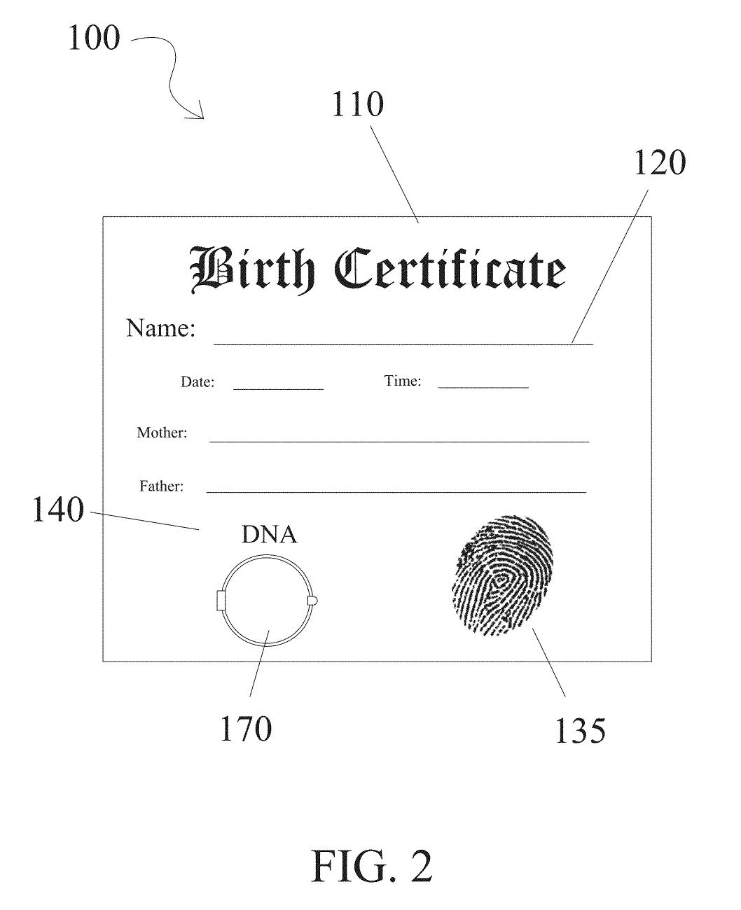

Now referring to FIG. 2, DNA birth certificate 100 is shown having a fingerprint 135 and a DNA sample container 170. DNA sample container 170 is attached to DNA birth certificate 100 and opens to allow the user to place a DNA containing biological sample within and then seal shut. Container 170 may be round as shown or any other suitable shape. Container 170 is relatively thin so that is does not stick out very far from the surface of DNA birth certificate 100, but again, other shapes could be used. Again, as discussed above, although fingerprint 135 is shown, other biometric identifiers could be used. Additionally, DNA certificate can be printed in a printer before attaching DNA sample container 170.

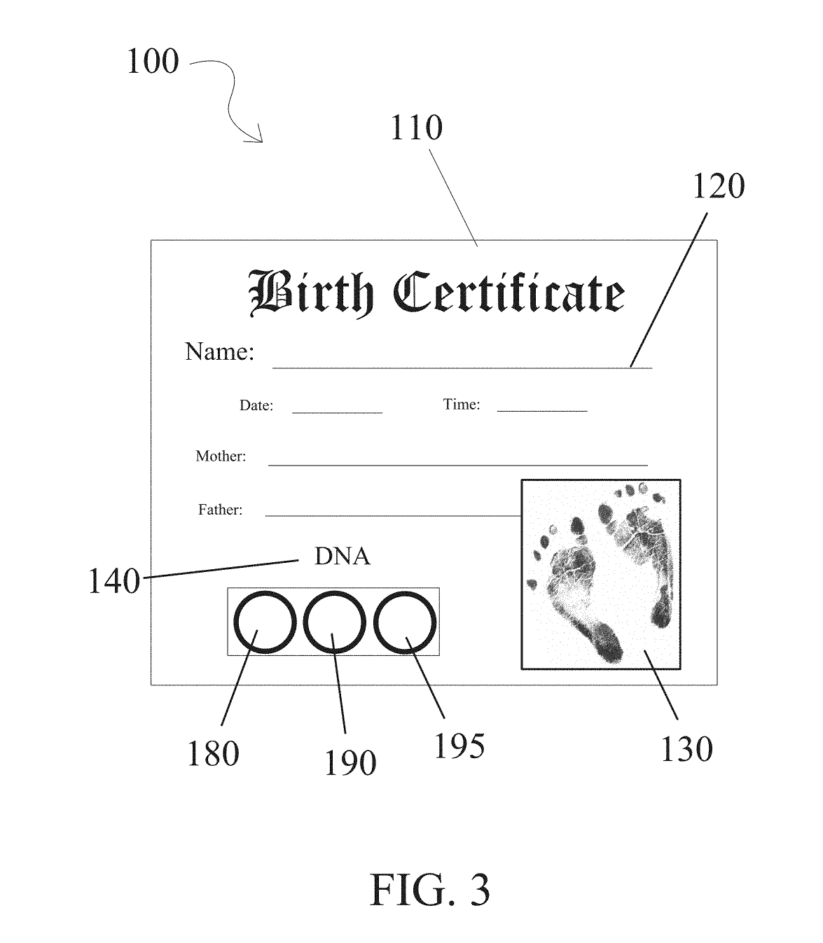

Referring now to FIG. 3, DNA birth certificate 100 is shown having a special DNA receiving section 140 with three DNA storage areas 180, 190 and 195 respectively. The material shown is known as WHATMAN FTA© Classic Card© and is the subject of several patents.

Referring to FIG. 17, Certificate 110 is shown with DNA sample area 140 having a sealable envelope 185 that may also hold DNA blood sample filter paper 165 and a disposable lancet. In use the user would carefully remove filter paper 165 and lancet 175, use lancet 175 to draw a small amount of blood from a finger, heel or other body area and then touch filter paper 165 to the blood and then place filter paper 165 back in sealable bag 185. Of course any DNA filter paper may be used as long as it allows a blood sample to be preserved therein as is known in the art. It allows for dry storage of DNA samples for years at room temperature without degrading. Of course other kinds of DNA storage materials may be used as long as they preserve the DNA without significant degradation as is known in the art. In some embodiments, sealable envelope 185 is empty and is not attached to certificate 110 until use by having an adhesive strip with a peelable portion (not shown) to attach it thereon as is known in the art.

Any one or all of DNA storage areas 180, 190 and 195 may be a sealable envelope to protect the DNA sample from environmental contamination and degradation. In the case where printing is desired before use, sealable envelopes or containers may be supplied with a peel off adhesive portion to allow attachment after printing. When using sealable envelopes or containers, care must be taken not to touch or contaminate the sample in any way and to ensure a complete and proper seal is achieved to protect the sample. In the case where a filter paper is used to store a blood sample, care must be used to insert the uncontaminated filter paper within the sealable envelope. If a blood sample is used, the user may be supplied with a lancet to draw the small amount of blood needed to impregnate the filter paper.

Additionally, an official certification proof area 225 is provided to allow an official stamp or other provenance may be attached to allow certificate 110 to be used to validate identity or other official requirements.

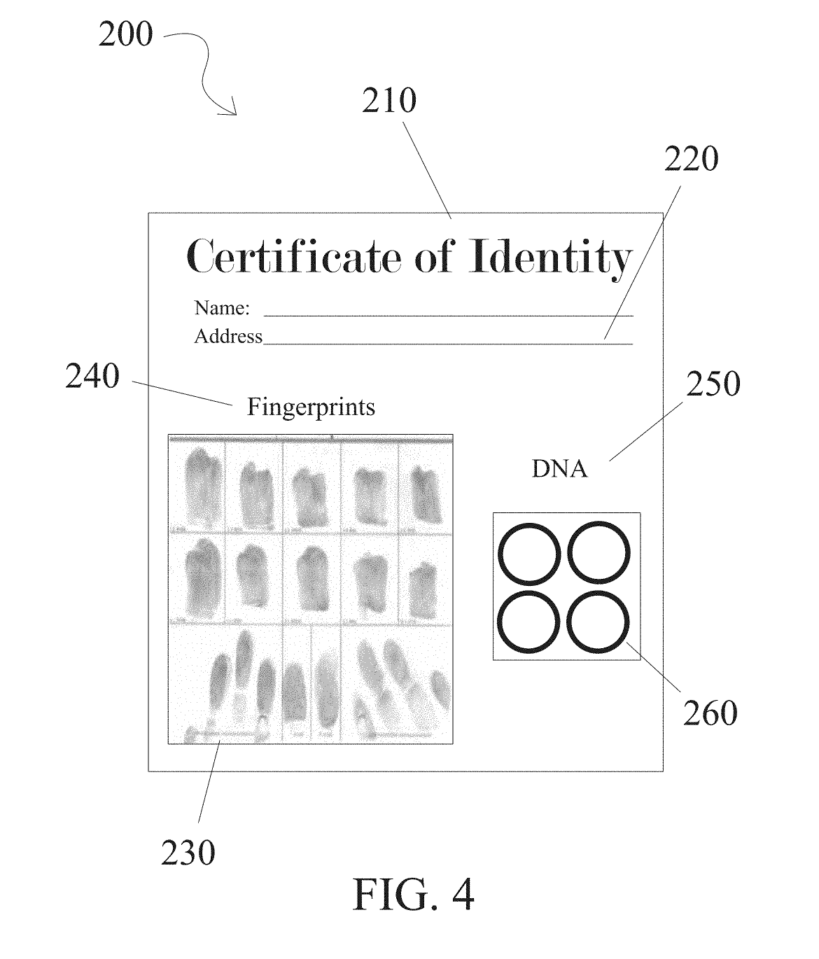

Referring now to FIG. 4, a DNA certificate of identity 200 is shown having a certificate 210 with written identification information 220 thereon. Although only name and address is shown, other kinds of identifying information may be provided such as age, weight, height, eye color, etc. to further help identify a person.

A biometric sample area 240 is shown having fingerprints 230. Again, other biometrics may be used such as physical samples, etc. In addition to fingerprints 230, a DNA sample area 250 is provided to store DNA samples on a FTA© CLASSIC CARD© 260. Again, other DNA preserving devices may be used as discussed above.

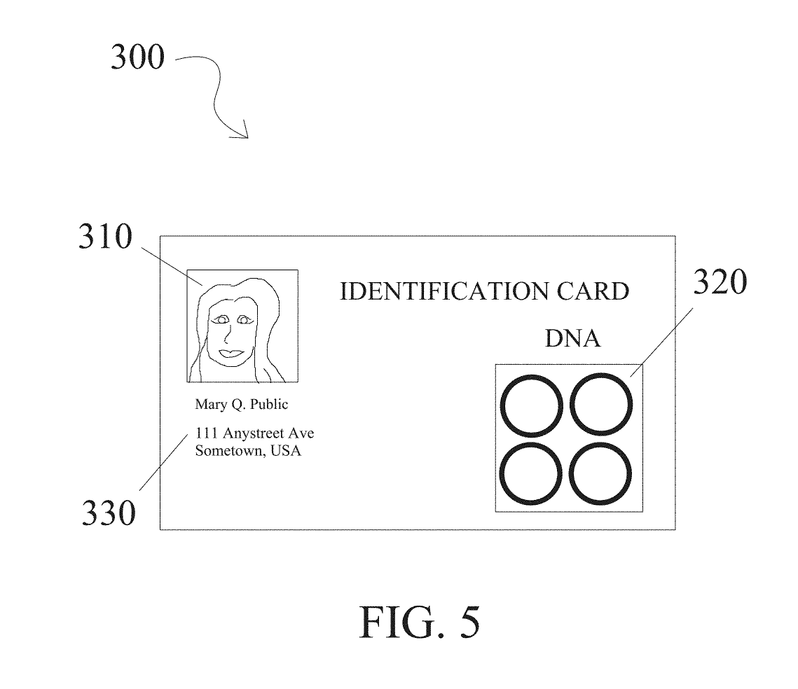

Now referring to FIG. 5, a DNA identification card 300 is shown having printed information 330 such as name and address, etc. In the embodiment shown, a picture 310 is provided to help identify a person. A DNA sample section 320 is provided to store DNA material that can be used to positively identify a person. Again, as discussed above, any suitable DNA storage method may be used to provide stable storage of DNA sample. DNA identification card 300 may be a state driver's license, sheriff's ID, etc.

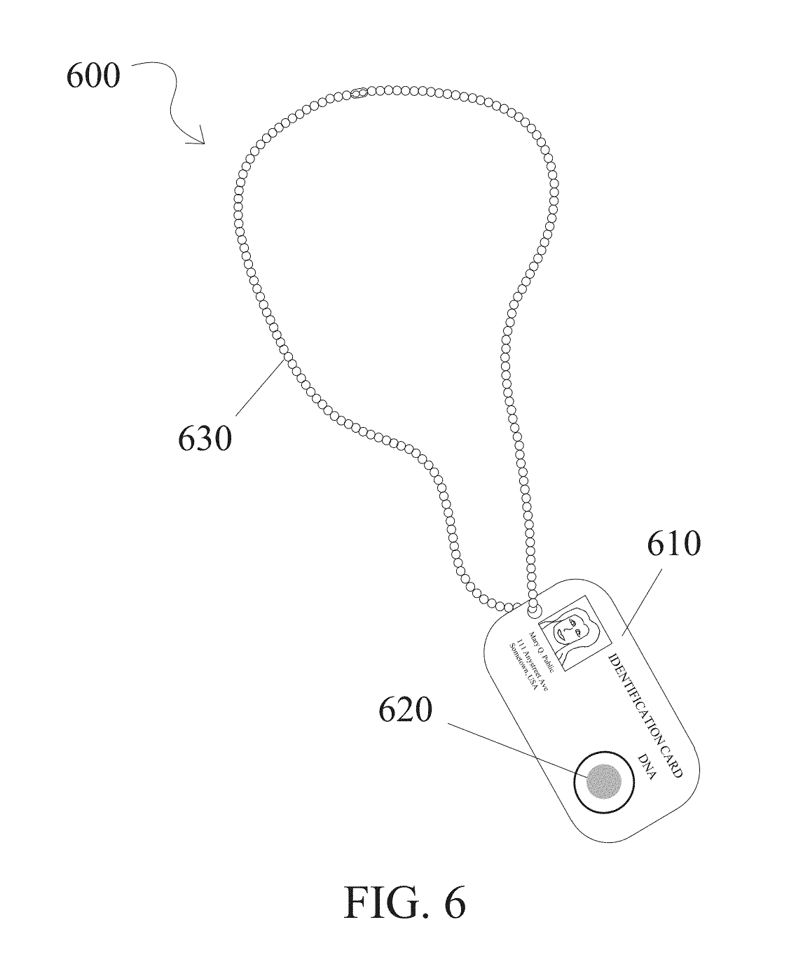

Referring now to FIG. 6, a DNA identification card 600 is shown having a chain or necklace portion 630 with an ID card 610. ID card 610 has a DNA preserving portion 620 as discussed above. Medical information may also be stored on ID card 610.

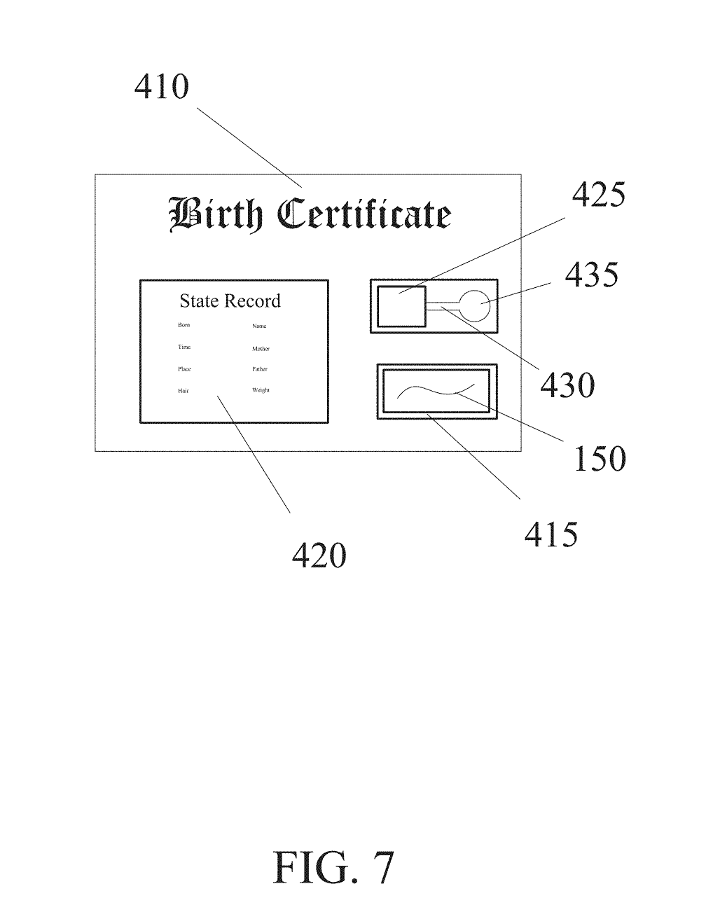

Now referring to FIG. 7, a biometric birth certificate 410 is shown having a printed information area 420 where official seal, birth information, etc. is recorded. In this embodiment, a DNA sequencing chip 425 is mounted on certificate 410 and a blood sample area 435 and blood path 425 is provided to transmit a blood sample to sequencing chip 425. A path 430 directs the blood sample to the sequencing chip 425. A DNA sample area 415 is provided to store and preserve a biological sample such as hair 150 or other sample. In this embodiment, an almost instant DNA sequence is analyzed and stored in chip 425 for future reference. Chip 425 contains a power source such as a small battery or solar generating panel (not shown) to power chip 425.

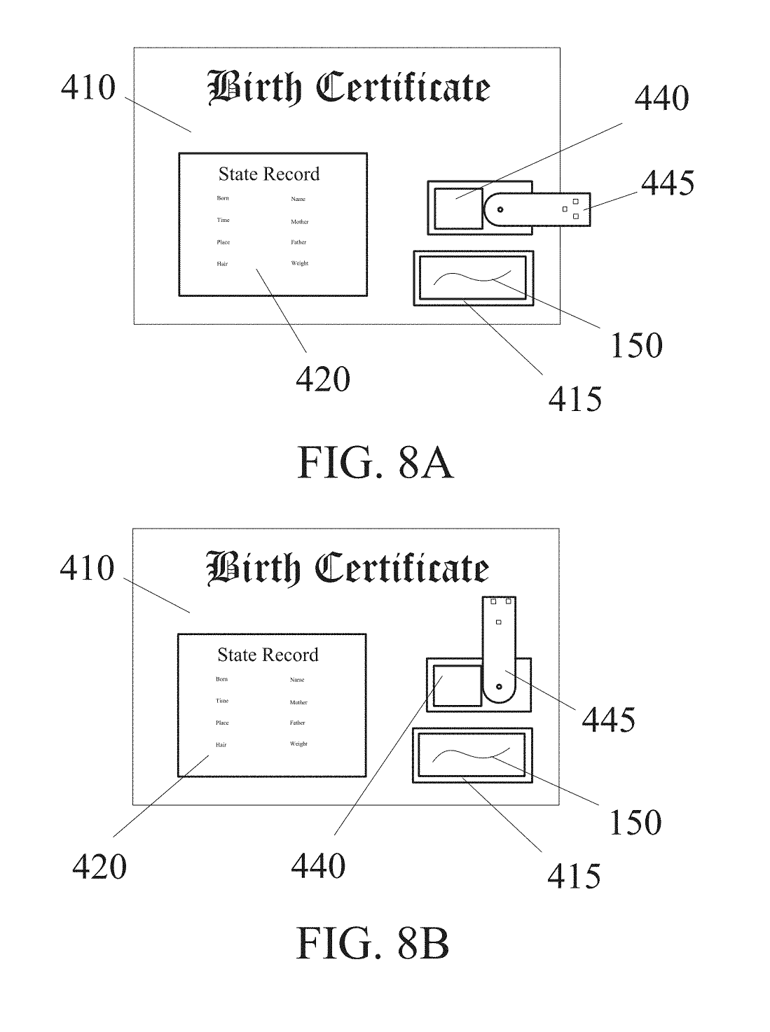

Referring to FIGS. 8A and 8B, the biometric birth certificate 410 is shown having a universal serial bus (USB) 445 connection built in certificate 410. USB connector 445 is connected to non-volatile memory chip 440 to record all the data including DNA results. FIG. 8B shows USB connector rotated to a storage position. Of course, other interface devices may be used, such as, but not limited to mini SD cards, flash sticks, memory cards, etc. In this embodiment, the DNA sample can be processed by a third party and the data uploaded and saved on biometric birth certificate.

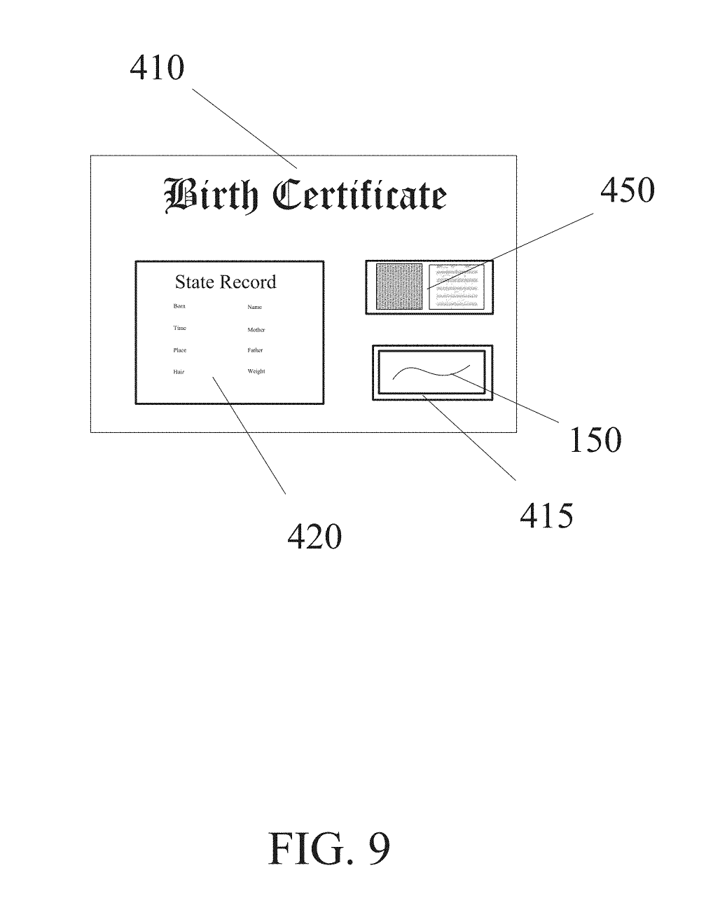

Now referring to FIG. 9, the biometric birth certificate 410 is shown having a DNA computer analysis printout storage section 450. The results may be stored in a compressed format such as a microfilm. In this embodiment, the DNA sample is processed in a laboratory and then the result is permanently stored on biometric birth certificate 410.

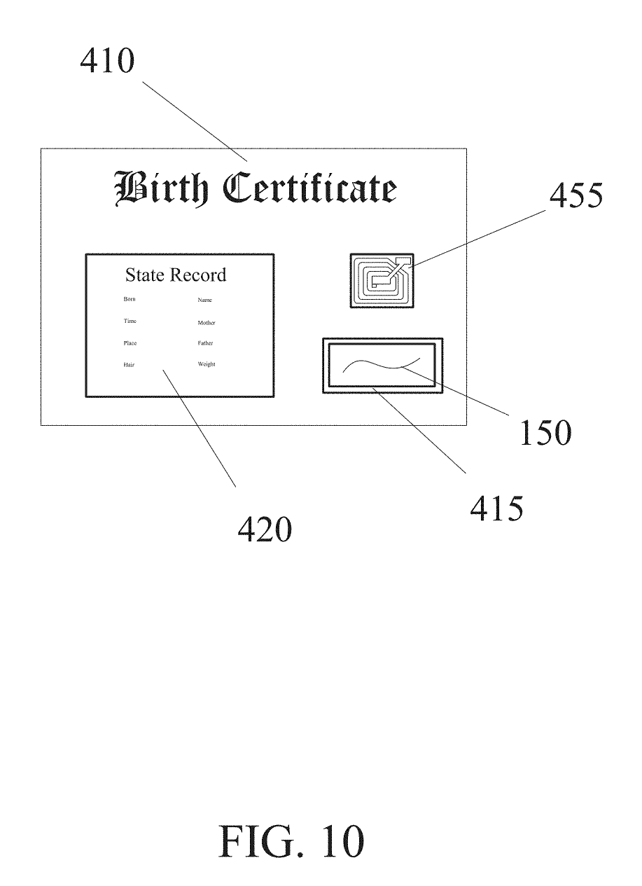

FIG. 10 shows a biometric birth certificate 410 with a radio-frequency identification and detection (RFID) chip 455 attached. Chip 455 stores data including DNA data that has been analyzed and prepared for uploading on to chip. 455. The data can then be stored and read out at a later date by an appropriate RFID reader as is known in the art.

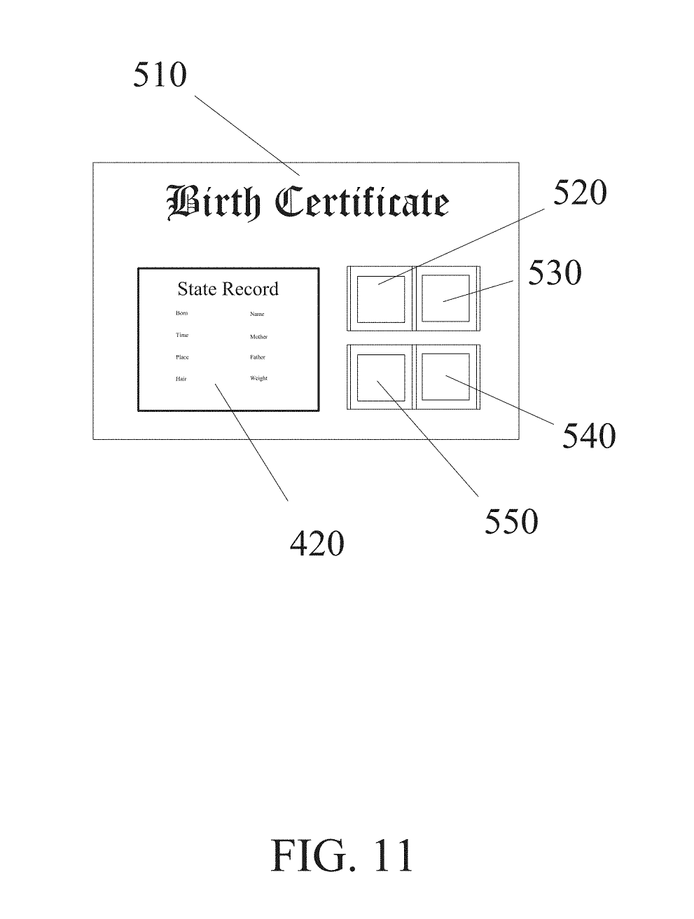

Referring to FIG. 11, a biometric birth certificate 510 is shown having a special area for a DNA sample from the mother 520, father 530, baby 550 and the baby's umbilical cord. This way an entire immediate family history of DNA is included in birth certificate 510 and provides access for any future needs. Sample areas 520, 530, 540 and 550 are sealed to prevent deterioration. They may be hermetically sealed and may be evacuated or filled with an inert gas such as nitrogen to further preserve the samples.

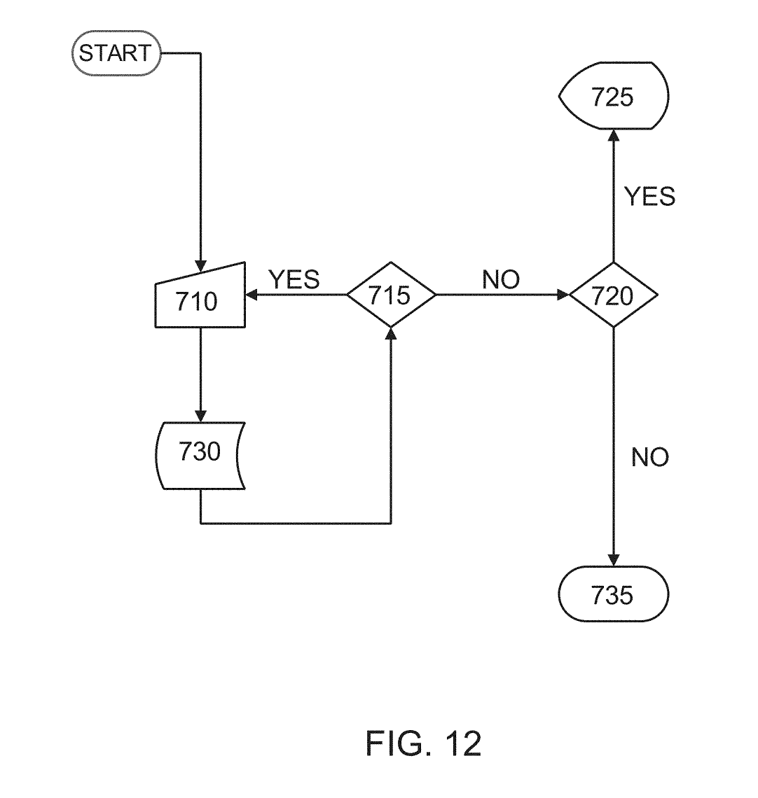

Referring to FIG. 12, a flow diagram illustrates the overall method of inputting, recording and retrieving DNA family history according to an embodiment of the present invention is shown starting by inputting DNA information or sample. The sample may include for example, a hair sample, fingernail, blood sample, etc. or other biological sample from which DNA information is extractable. Alternatively, the information may be electronic where the analysis is prepared earlier and only the DNA results are recorded. Additionally, the input may be done electronically such as by Wi-Fi, Bluetooth, an internet connection or other electronic transmission means and could be part of an automated process based on a subscriber model.

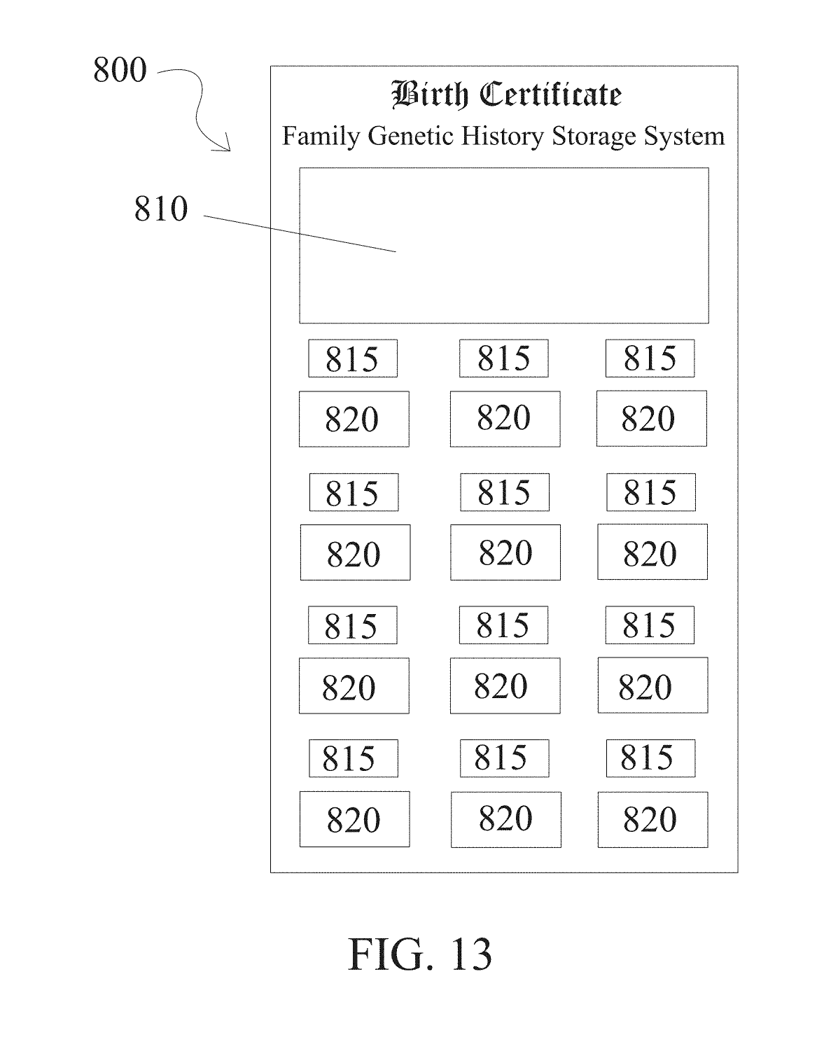

The data is stored 730 either electronically as in the case of electronic DNA data or a physical sample container 820 such as the ones shown in FIG. 13. In a query process 715, either more data is stored by inputting additional data 710 or passed to another query process 720 where either the data is retrieved and or displayed 725 or the process ends 735. In this way, a family history of DNA data is built up and made available for future use.

Referring now to FIG. 13, a birth certificate and family genetic history storage system sample holding card 800 is shown having an official information area 810 where any official information and authentication information may appear. Ordinarily this portion is filled out according to specific state requirements and may contain hologram, raised stamp or other authenticating scheme to ensure that the information is authentic. In the sample area a plurality of heading portions 815 are provided to display the familial relationship of the sample stored there.

Again, the data may be physical or electronic. In this way, as much family data is preserved as possible by merely continuing to input available family information. The advantage of physical samples is that DNA analysis can be expensive and in the future it may be much more economical to sequence the information than presently available. Of course some of the disadvantages of storing physical samples is the possibility of contamination and requires more physical space. By having a plurality of storage spaces 820 available, it encourages users to collect the data before it is needed and often too late to collect.

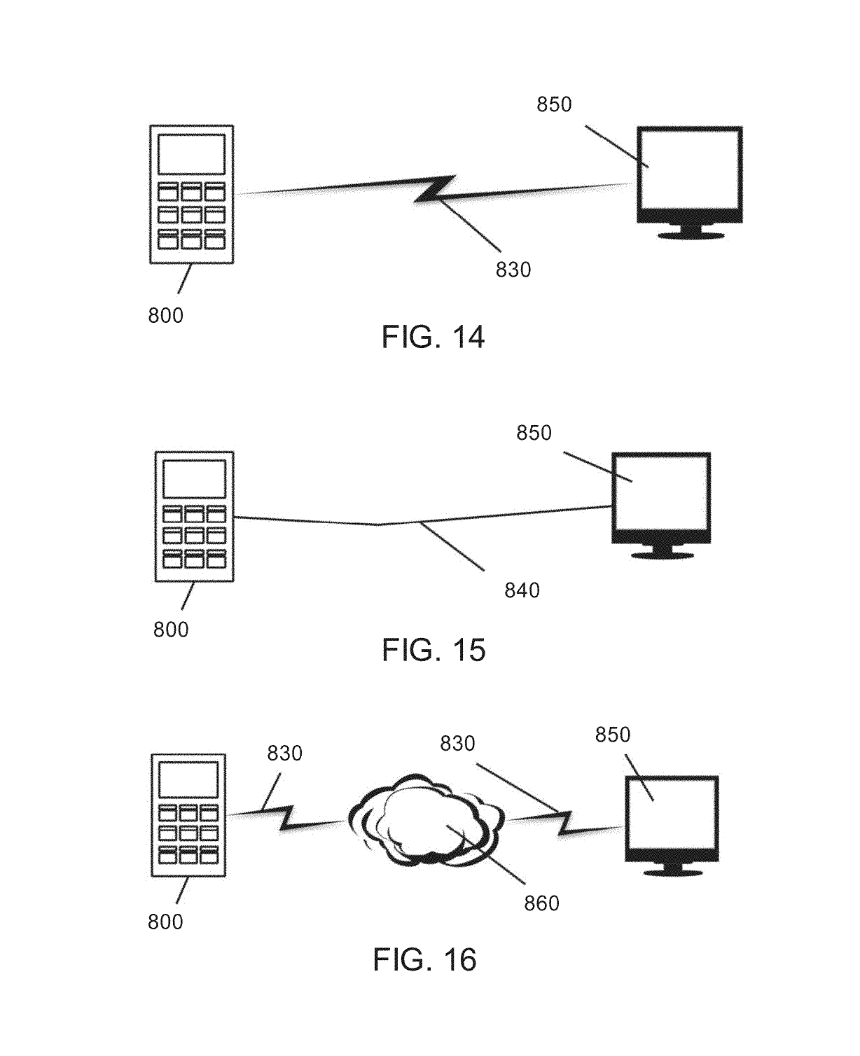

Referring now to FIG. 14, an electronic storage embodiment is shown having a birth certificate and family genetic history storage system sample holding card 800 that is interfaced through a wireless connection 830 and a display device 850. Wireless connection 830 may be a Wi-Fi, Bluetooth or any other suitable wireless interface. Display device 850 may be a computer, tablet, smart phone, etc. or any other suitable display device as is known in the art.

Referring now to FIG. 15, an electronic storage embodiment is shown having a birth certificate and family genetic history storage system sample holding card 800 that is interfaced through a wired connection 840 and a display device 850. Wired connection 840 may be a LAN, WAP or other suitable network as is known in the art. Display device 850 may be a computer, tablet, smart phone, etc. or any other suitable display device as is known in the art.

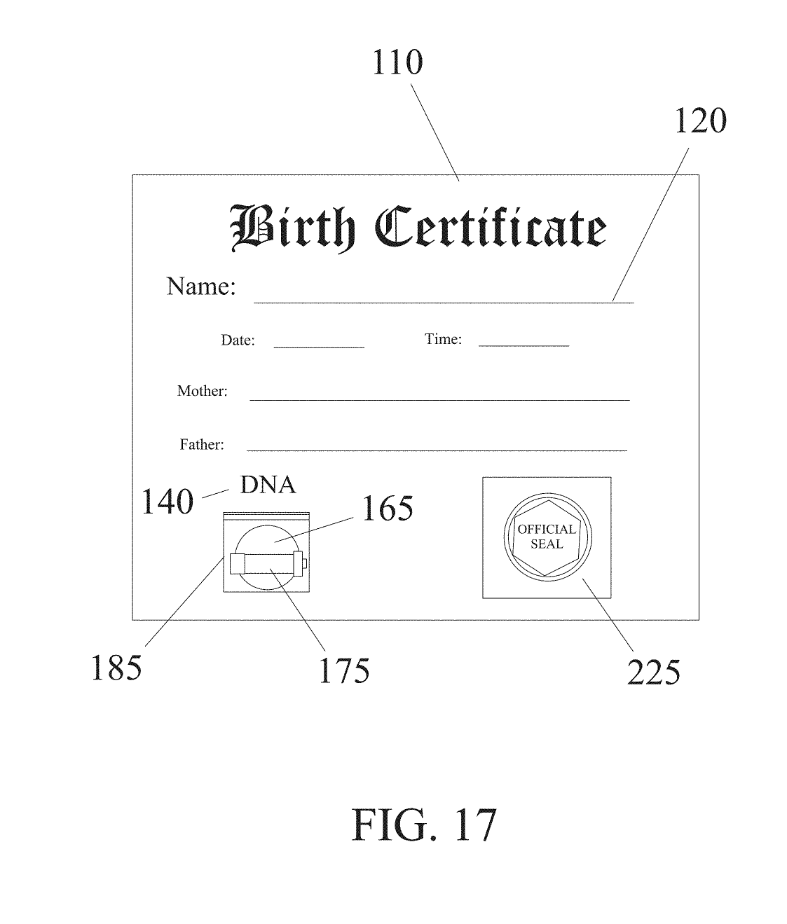

Referring now to FIG. 16, an electronic storage embodiment is shown having a birth certificate and family genetic history storage system sample holding card 800 that is interfaced through a wireless connection 830 which connect to a cloud based service 860 and a display device 850. Wireless connection 830 may be a Wi-Fi, Bluetooth or any other suitable wireless interface. Display device 850 may be a computer, tablet, smart phone, etc. or any other suitable display device as is known in the art.

Although the instant invention has been described in relation to particular embodiments thereof, many other variations and modifications and other uses will become apparent to those skilled in the art.

What is claimed is:

1. A family genetic history storage system for storing DNA information on an identification certificate comprising:
said identification certificate having a selected area for attaching an identification indicators;
at least one DNA blood sample storage area;
said at least one DNA blood sample storage area wherein a blood sample is stored and preserved for future use; and
a means for isolating and storing said blood sample from environmental contamination.

2. The family genetic history storage system for storing DNA information on an identification certificate according to claim 1 wherein said means for isolating and storing said blood sample is a sealable envelope.

3. The family genetic history storage system for storing DNA information on an identification certificate according to claim 1 wherein said means for isolating and storing said blood sample is a sealable container.

4. The family genetic history storage system for storing DNA information on an identification certificate according to claim 2 further comprising a single use sterile blood lancet removably disposed within said sealable envelope.

5. The family genetic history storage system for storing DNA information on an identification certificate according to claim 2 further comprising a DNA blood sample filter paper removably disposed within said sealable envelope.

6. The family genetic history storage system for storing DNA information on an identification certificate according to claim 1 further comprising an official certification proof area whereby official provenance is attached to certify authenticity.

7. A family genetic history storage system for storing DNA information on an identification certificate comprising:
said identification certificate having a selected area for attaching an identification indicators;
at least one DNA sample storage area;
said at least one DNA sample storage area wherein a DNA sample is stored and preserved for future use; and
a means for isolating and storing said DNA sample from environmental contamination.

8. The family genetic history storage system for storing DNA information on an identification certificate according to claim 7 wherein said DNA sample is a DNA blood sample.

9. The family genetic history storage system for storing DNA information on an identification certificate according to claim 7 wherein said DNA sample is a biological cell sample.

10. The family genetic history storage system for storing DNA information on an identification certificate according to claim 8 wherein said means for isolating and storing said DNA sample is a sealable envelope.

11. The family genetic history storage system for storing DNA information on an identification certificate according to claim 9 wherein said means for isolating and storing said blood sample is a sealable container.

12. The family genetic history storage system for storing DNA information on an identification certificate according to claim 10 further comprising a single use sterile blood lancet removably disposed within said sealable envelope.

13. The family genetic history storage system for storing DNA information on an identification certificate according to claim 10 further comprising a DNA blood sample filter paper removably disposed within said sealable envelope.

14. The family genetic history storage system for storing DNA information on an identification certificate according to claim 7 further comprising an official certification proof area whereby official provenance is attached to certify authenticity.
